(19) United States
(12) Patent Application Publication
(10) Pub. No.: US 2015/0246074 A1
(43) Pub. Date: Sep. 3, 2015

(54) METHODS OF ATTENUATING OPIOID TOLERANCE

(71) Applicant: The Board of Trustees of the University of Illinois, Urbana, IL (US)

(72) Inventors: Tingyu Qu, Chicago, IL (US); Jianguo Cheng, Solon, OH (US); Jinhua Sun, Hangzhou (CN); Hongna Yang, Jinan (CN); Kathleen Cheng, Solon, OH (US); Jun Shen, Newton, MA (US)

(21) Appl. No.: 14/635,479

(22) Filed: Mar. 2, 2015

Related U.S. Application Data
(60) Provisional application No. 61/945,890, filed on Feb. 28, 2014.

(51) Int. Cl. A61K 35/28; A61K 31/485; A61K 9/00; C12N 5/0775

(52) U.S. Cl. CPC A61K 35/28; C12N 5/0663; A61K 31/485; A61K 9/0085; A61K 2035/124

(57) ABSTRACT

Disclosed are methods of reducing pain and/or preventing or reducing opioid tolerance in a subject by administering to the subject mesenchymal stem cells.

### CROSS REFERENCE TO RELATED PATENTS

This application claims the benefit of U.S. Provisional Application No. 61/945,890, filed Feb. 28, 2014, which is incorporated by reference in its entirety.

### STATEMENT REGARDING FEDERALLY SPONSORED RESEARCH OR DEVELOPMENT

Not applicable.

### FIELD OF THE INVENTION

The present invention relates generally to methods of using mesenchymal stem cells to treat pain and reduce opioid tolerance.

### BACKGROUND OF THE INVENTION

Millions of Americans suffer from chronic pain, at a cost of hundreds of billions of dollars in terms of medical expenses and lost productivity. Pain is a leading cause of short- and long-term disability among active duty and retired military personnel. Two out of every three U.S. armed forces veterans have persistent pain attributable to military service. Ineffective pain treatment leads to pain-related impairments, occupational disability, and medical and mental complications with long-term costs to the military health and disability systems, and to society at large.

Opioids can be efficacious in the treatment of pain, and cost-effective analgesics. However, the development of opioid tolerance over time can limit opioid effectiveness, compromise safety, and lead to detrimental consequences, including drug overdose, abuse, and addiction. Tolerance is a physiologic process in which the body adjusts to medications that are administered frequently. Patients who develop a tolerance to an opioid analgesic often require higher doses of the analgesic to achieve pain relief. High doses of opioids can cause cognitive impairment, respiratory depression, nausea and vomiting, constipation, reduced communication capacity, diminished quality of life, over sedation, and death. Deaths caused by opioid prescription drug abuse in the US have increased dramatically in recent years. The Center for Disease Control and Prevention (CDC) has declared the problem an ongoing “national epidemic”.

Effective strategies to treat opioid tolerance have not been discovered despite the extensive efforts devoted to this area of research, primarily because the exact mechanisms of opioid tolerance are poorly understood.

There is a need in the art for methods for countering opioid tolerance. The present invention addresses that need.

### SUMMARY OF THE INVENTION

In an aspect, this invention is a method of treating pain in a subject comprising administering an effective pain-reducing number of mesenchymal stem cells to the subject.

In another aspect, this invention is a method of treating pain in a subject comprising administering mesenchymal stem cells to the subject and administering one or more opioids to the subject wherein the number of administered mesenchymal stem cells and opioids is effective to mitigate pain in the subject.

In another aspect, this invention is a method of attenuating opioid tolerance in a subject comprising administering a number of mesenchymal stem cells effective to attenuate opioid tolerance into the subject before or during the course of opioid treatment.

In certain embodiments of the invention, the mesenchymal stem cells are harvested or isolated from bone marrow.

In other embodiments, the bone marrow is the subject's bone marrow.

In other embodiments, the mesenchymal stem cells are expanded in cell culture after harvesting from bone marrow.

In other embodiments, the mesenchymal stem cells are transplanted into the subject's intrathecal space.

In other embodiments, the opioid is morphine.

In other embodiments, the mesenchymal stem cells are transplanted into the subject prior to treating with the opioids.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed for carrying out this invention, but that the invention will include all embodiments falling within the scope of the present application.

### BRIEF DESCRIPTION OF THE DRAWINGS

FIG. 1 shows the effect of morphine on cyclic adenosine monophosphate (cAMP) levels in SH-SY5Y cells.

FIG. 2 shows the time course of the development of cAMP upregulation in SH-SY5Y cells.

FIG. 3A shows that upregulation of cAMP induced by morphine in SH-SY5Y cells was significantly attenuated by cell-to-cell contact with hMSCs.

FIG. 3B shows that shows that upregulation of cAMP induced by morphine in SH-SY5Y cells was not significantly attenuated without cell-to-cell contact with hMSCs.

FIG. 4 shows the analgesic and anti-tolerance effects of stem cell transplantation determined by the withdrawal thresholds to mechanical stimulation.

FIG. 5 shows the analgesic and anti-tolerance effects of stem cell transplantation determined by the withdrawal thresholds to thermal stimulation.

### DETAILED DESCRIPTION OF THE INVENTION

It was recently recognized that opioids can cause activation of microglial cells and astrocytes in the spinal cord and brain, leading to neuroinflammation in the central nervous system, which is directly associated with opioid tolerance. Reducing neuroinflammation may therefore be an effective strategy to attenuate opioid tolerance and enhance the analgesic effect of opioids.

Mesenchymal stem cells (MSCs) from bone marrow were evaluated for the ability to reduce opioid tolerance and enhance analgesia, as MSCs, as MSCs are recognized to have anti-inflammatory and immunomodulatory effects. As described below, MSCs were transplanted into the intrathecal space surrounding the spinal cord and brain of morphine tolerant rats and were found to reduce morphine tolerance and enhance analgesia in rats. This strategy is clinically relevant because MSC transplantation has been safely used in clinical practice to treat many diseases such as leukemia. Transplantation of MSCs, particularly autologous MSCs obtained from the bone marrow of the patient, to treat chronic pain and opioid tolerance is an innovative and practical approach. MSCs can be readily obtained, are safe to transplant, and can circumvent potential immune rejection. This new concept was tested, as described in the Examples, to develop an innovative solution to the problem of opioid tolerance and provide an important advance in the management of chronic pain. This approach would fundamentally reduce the need for the administration of increasing amounts of opioids to treat pain and reduce the risk of opioid side effects, abuse, and addiction.

This invention, therefore, relates to methods of using transplanted MSCs to treat pain and/or attenuate or reduce opioid tolerance in a subject.

The terms “isolate”, “isolated” and “harvested” generally refer to a mesenchymal stem cell which has been separated from its natural environment. This term includes gross physical separation from its natural environment, e.g., removal from the donor animal. In certain embodiments, an isolated cell is not present in a tissue, i.e., the cell is separated or dissociated from the neighboring cells with which it is normally in contact or associated. Preferably, cells are administered as a cell suspension. As used herein, the phrase “cell suspension” includes cells which are in contact with a medium and which have been dissociated, e.g., by subjecting a piece of tissue to gentle trituration.

In certain embodiments, the population of isolated mesenchymal stem cells is expanded by culturing in a growth medium. The term “growth medium” generally refers to a medium sufficient for the culturing of bone marrow-derived cells. In particular, one medium for the culturing of the cells of the invention comprises Dulbecco's Modified Essential Media (DMEM). The DMEM may be supplemented with serum, most preferably fetal bovine serum or human serum. In some cases different growth media are used or different supplementations are provided. In certain chemically-defined media the cells may be grown without serum present at all. In such cases, the cells may require certain growth factors, which can be added to the medium to support and sustain the cells. Factors to be added for growth in serum-free media may include one or more of bFGF, EGF, IGF-I, and PDGF.

An “effective amount” refers to a concentration, amount, or number of MSCs or an amount or volume of a composition comprising MSCs sufficient to achieve a desired result, for example, treating chronic pain and/or attenuating opioid tolerance. The number of MSCs that constitute an “effective amount” may vary depending on various factors, including, for example, the severity of the pain, the condition, weight, or age of the patient to be treated, the frequency of dosing, or the route of administration, but can be determined routinely by one of ordinary skill in the art. With respect to the number of cells as administered to a patient, an effective amount is generally about 10⁴ cells/kg body weight or more, and may range from about 10⁴cells/kg body weight to about 10⁶ cells/kg body weight. In specific embodiments, an effective number may range from about 10⁵ to about 10⁸ cells/kg body weight.

“Opioid tolerance” refers to a progressive reduction in a subject's reaction to an administered opioid drug such that an increase in the amount of the opioid drug administered is required to achieve the desired effect. The number of MSCs effective to “attenuate” or “reduce” opioid tolerance refers to an amount of transplanted MSCs sufficient to allow effective pain relief over time without the need for substantially increasing the dosage of opioid drug administered, i.e., the dose of the opioid required to achieve the desired analgesic effect remains relatively constant over time.

For all embodiments of the invention, a subject having chronic pain is administered a population of MSCs in an amount effective to treat the pain and/or attenuate opioid tolerance. Compositions administered to the subject typically include the population of MSCs and a pharmaceutically acceptable carrier. The MSC administration may be accomplished by any means including catheter, syringe, shunt, stent, microcatheter, and the like.

For instance, in one embodiment, the population of MSCs is administered by direct stereotaxic injection, e.g., needle. The needle may be any size to facilitate movement of cells through the hollow bore. The needle may be inserted directly through the skin to the tissue site of interest, or alternatively the needle may be used with a device to ease guidance of the needle to the tissue site, such as, for example, a guide wire. The needle and guidance device can be either preassembled or delivered to the trained practitioner; the trained practitioner may assemble the device just prior to or during use.

The MSCs can be administered in any physiologically compatible carrier, such as a buffered saline solution. Pharmaceutically acceptable carriers and diluents discussed within this disclosure, including but not limited to, saline, aqueous buffer solutions, solvents and/or dispersion media. The use of such carriers and diluents is well known in the art. Other examples include liquid media such as Dulbecco's modified eagle's medium (DMEM), sterile saline, sterile phosphate buffered saline, Leibovitz's medium (L15, Invitrogen, Carlsbad, Calif.), dextrose in sterile water, and any other physiologically acceptable liquid. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosol, and the like. Solutions of the invention can be prepared by using a pharmaceutically acceptable carrier or diluent and, as required, other ingredients enumerated above, followed by filtered sterilization, and then incorporating the population of cells as described herein.

Pharmaceutical compositions of the invention may include preparations made from cells that are formulated with a pharmaceutically acceptable carrier or medium. Suitable pharmaceutically acceptable carriers include any discussed within this disclosure, including but not limited to, water, salt solution (such as Ringer's solution), alcohols, oils, gelatins, polyvinyl pyrrolidine, carbohydrates such as lactose, amylose, or starch, fatty acid esters, and hydroxymethylcellulose. Such preparations can be sterilized, and if desired, mixed with auxiliary agents such as lubricants, preservatives, stabilizers, wetting agents, emulsifiers, salts for influencing osmotic pressure, buffers, and coloring agents. Pharmaceutical carriers suitable for use in the present invention are known in the art and are described, for example, in Pharmaceutical Sciences (17^{th} Ed., Mack Pub. Co., Easton, Pa.) and WO 96/05309.

The foregoing may be better understood by reference to the following experiments, which are presented solely for purposes of illustration and are not intended to limit the scope of the invention.

Determination of the Anti-Tolerance Effects of hMSCs on Repeated Morphine Administration

The potential effect of human mesenchymal stem cells (hMSCs) on the development of morphine-induced tolerance was investigated in vitro by a co-culture system of hMSCs and the neuronally-differentiated SH-SY5Y cells, and in vivo by spinal transplantation of rat MSCs (rMSCs) in rat pain models.

### Materials and Methods

Culture of SH-SY5Y Cells. SH-SY5Y cells, a well-established stable human neuroblastoma cell line, were purchased from American Type Culture Collection (ATCC, Manassas, Va.) and grown in Dulbecco's modified Eagle's medium (DMEM) with 10% fetal bovine serum, 100 μU/ml penicillin, and 100 μg/ml streptomycin at 37° C. in a 5% CO₂ humidified incubator (Fisher). Before experiments, SH-SY5Y cells were treated with 10 μM retinoic acid (RA) for 6 days to induce neuronal differentiation. These differentiated SH-SY5Y cells were then treated with morphine (10 μM) for 24 h unless otherwise specified.

Culture of hMSCs and HDFs. Human bone marrow samples were purchased from AllCells LLC (Emeryville, Calif.). Cells were isolated from bone marrow tissue by combining the tissue with Dulbecco phosphate-buffered saline (DPBS; Gibco) and centrifuging at 500×g for 10 min at 20° C. The cells were resuspended and gently layered onto a Percoll cushion (density, 1.073 g/mL; Invitrogen, Carlsbad, Calif.). The low-density hMSC-enriched mononuclear fraction was collected, washed with DPBS, and centrifuged. The hMSCs were cultured using previously described methods. In brief, hMSCs were plated in 75-cm² culture flasks at a concentration of 1×10⁵ cells/cm² in 20 ml of serum-supplemented growth medium including DMEM, penicillin-streptomycin mixture (1:100, Invitrogen, Carlsbad, Calif., USA), and 10% FBS (Stem Cell Technologies, Vancouver, BC, Canada), incubated at 37° C. in a 5% CO₂ humidified incubation chamber, and fed by replacing half of the culture media twice a week. Cells were passaged by incubating with 0.05% trypsin-EDTA (Gibco) for 5 min at room temperature to gently release the cells from the surface of the culture flask after reaching about 80% confluency. Culture medium was added to stop trypsinization, and the cells were centrifuged at 350×g for 5 min at room temperature, re-suspended, and transferred into new culture flasks at a concentration of 1×10⁵ cells/cm2 for continuous culture and expansion to reach a sufficient number of cells. hMSCs with five or fewer passages are designated early passage, while hMSCs >passage 10 were defined as the late passage. Primary human dermal fibroblasts (HDFs) were purchased from Cell Applications, Inc. (San Diego, Calif.) and cultured in 1640 medium containing 10% FBS, 1% L-glutamine, 1% penicillin/streptomycin at 37° C. in a humidified atmosphere of 5% CO₂.

Co-culture of SH-SY5Y Cells, hMSCs, and HDFs. Co-cultures of SH-SY5Y cells and hMSCs were performed using cell culture inserts with 0.4 μm diameter pores according to the manufacturer's protocol (Greiner Bio-One, Monroe, N.C.). Briefly, 100 μl aliquots of cell suspension containing different concentrations of hMSCs were pipetted onto the membranes of the cell culture inserts or the membrane of the inverted cell culture insert and the cells were allowed to adhere overnight at 37° C. in a 5% CO₂ incubator. Subsequently, the cell culture insert was placed into the well of a 12 well plate pre-seeded with differentiated SH-SY5Y cells, which were pretreated with 10 μM morphine for 24 h. Co-cultures of SH-SY5Y cells and hMSCs were maintained for additional 36 hours in the presence of 10 μM morphine at 37° C. in a 5% CO₂ incubator. The co-cultures of SH-SY5Y cells and HDFs of early passages (P≦5) were performed by using the same method described for co-cultures of SH-SY5Y cells and hMSCs at identical conditions.

cAMP Accumulation Assay. Levels of cAMP, which is involved in a cellular mechanism of opioid tolerance development, were determined in morphine-treated SH-SY5Y cells. Thirty-six hours after co-culturing with hMSCs or HDFs, SH-SY5Y cells were harvested with a non-enzymatic cell dissociation solution and washed once with HBSS buffer. cAMP accumulation in SH-SY5Y cells was assayed using a LANCE™ cAMP kit (PerkinElmer, Waltham, Mass.) according to the manufacturer's protocol. SH-SY5Y cells were centrifuged at 1000 g for 2 min, resuspended at a concentration of 2×10⁶ cells/ml in stimulation buffer (1×HBSS, 5 mM HEPES, 0.1% BSA, 0.5 mM IBMX, pH 7.4) and mixed with 50 μM forskolin. Alexa pluor® 647 labeled antibody was added to the cell suspension and the cells were incubated at 37° C. for additional 15 min, and detection mix was then added to the final cell suspension. The cell sample was further incubated for 1 h in the dark and read on a TECAN instrument (San Jose, Calif.) to measure the LANCE signal. The LANCE signal obtained at 665 nM was directly used to analyze the cAMP levels. The signal at 615 nM was used to identify dispensing or quenching problems. The cAMP standard curve was assayed according to the manufacturer's instructions. The cAMP formation was calculated as the percentage of forskolin-stimulated cAMP accumulation without morphine, which was defined as 100%. All data were expressed as mean±SD. Statistical comparisons were analyzed using Prism software. Values of p<0.05 were considered statistically significant.

### Analgesic and Anti-Tolerance Effects of Stem Cell Transplantation

Animal care and use. The experimental protocols were approved by the Institutional Animal Care and Use Committee of Cleveland Clinic (IACUC #: 2010-0460). Experiments were performed on 24 male Sprague-Dawley rats (250-300 g), purchased from Harlan Laboratories (Indianapolis, Ind.). The animals were housed in group cages (2 per cage), allowed free access to food and water, and maintained on a 12/12 hour light/dark cycle. Rats that exhibited excessive circling or aggressive behaviors were removed from the group cages and housed separately.

Experimental design. Animals were habituated daily to the experimental environment for 1 week before testing. Pain sensitivity to mechanical or thermal stimuli applied to the hindpaws was determined by the thresholds of withdrawal of the hindlimbs from the stimuli. Transplantation of MSCs or injection of PBS as control was performed in the end of the second week after the baseline thresholds of the hindlimb withdrawal response had been established. Daily morphine injections were conducted to induce tolerance in weeks 4 and 5. The effects of MSC transplantation or PBS injection on morphine-induced tolerance were evaluated every other day. All tests and injections were performed in the morning. Twenty-four rats were randomly divided into two groups receiving either MSC transplantation (MSCs group, n=12) or PBS injection as control (PBS group, n=12). Six rats in each group received either mechanical or thermal stimulation to determine the withdrawal thresholds.

Anesthesia. Brief anesthesia was achieved using sevoflurane (Abbott Laboratories, North Chicago, Ill.) to facilitate short procedures. Sevoflurane (2-3 ml) was added to a gauze pad and placed on the bottom of a 4 liter sealed plastic chamber. The gauze pad was encased in a barrier to prevent direct contact with the rat placed inside the chamber. The lid was tightly sealed until the animal rolled onto its side with reduced respiration rates. The rat was immediately removed from the chamber for short procedures. After the procedure, the animal was placed into a recovery cage and monitored until it was fully awake and able to move freely. The procedure was performed in a hood that vented to the outside to protect the laboratory personnel who also wore a mask.

Stem cell transplantation. Mesenchymal stem cells were isolated from the bone marrow of Sprague-Dawley rats (200-250 g). The primary cells were dissociated by 0.25% trypsin (Invitrogen) and cultured to MSCs. The cells of passage 2 or 3 were used for transplant. Immediately before transplantation, the cells were gently dissociated with 0.25% Trypsin, pelleted by centrifugation, counted by trypan blue exclusion, and suspended in 50 μl of PBS.

Approximately 5×10⁵ MSCs in 50 μl PBS were transplanted into the intrathecal space filled with cerebrospinal fluid (CSF) surrounding the lumbar spinal cord. Briefly, the animals were anesthetized with sevoflurane and placed on a rolled pad so that lumbar spine was arched. The skin was shaved and disinfected with 70% isopropyl alcohol. The L4-L5 or L5-L6 lumbar interspace was identified by palpating and counting the spinous processes. A 0.5-inch 30-gauge needle (Becton-Dickinson, Franklin Lakes, N.J.) connected to a Hamilton syringe filled with MSCs in 50 μl PBS was slowly advanced through the tissues between the interspace. When the needle reached the intrathecal or subarachnoid space, a sudden loss of resistance along with a tail flick was typically observed. Positive aspiration of CSF was used to confirm the needle placement. The cells were slowly injected and the needle was carefully removed. The same procedure was performed in the control animals, except that 50 μl PBS containing no MSCs was injected.

Morphine injection. Morphine tolerance was induced by repeated daily injections for 2 weeks. All rats (n=24) received daily subcutaneous injections of morphine (7.5 mg/kg) in weeks 4 and 5, one week after MSC transplantation or PBS injection. The injections were performed on conscious rats that had been habituated to handling by the experimenters.

Behavioral tests. Withdrawal of the hindlimbs in response to mechanical or thermal stimuli to the hindpaws was used as a measure of pain sensitivity. Withdrawal thresholds were quantified as the mechanical pressure in grams applied to the hindpaws through calibrated filaments (von Frey filaments) or the time interval in seconds between the activation of an infrared thermal stimulation and the withdrawal of the limb. A low threshold indicates high pain sensitivity. Rats were habituated to the experimental environment to minimize stress before behavioral testing and experimental procedures. The behavioral measurements were performed prior to transplantation to determine the baseline values and after the transplantation at regular intervals to determine the analgesic and anti-tolerance effects of the transplantation. All behavioral tests were performed by experimenters who were blinded to the treatments for objective assessment.

Mechanical pain sensitivity. The withdrawal thresholds to mechanical stimulation were determined using a Semmes-Weinstein von Frey Touch Test Sensory Evaluator (North Coast Medical, Inc. Morgan Hill, Calif.). The animal was loaded into a plexi-glass chamber, resting on a 6 mm wire grid unrestrained. After a 15 min acclimation period, the plantar surface of the footpads of the hindpaws was stimulated with the calibrated von Frey filaments. The filaments were first applied in ascending order of force (0.4-60 grams) until they bent for ˜3 seconds or until foot withdrawal took place. The filaments were then applied in descending order, beginning with the next lighter filament until there was no withdrawal response. The threshold was defined as the lightest filament to evoke a withdrawal. The procedure was repeated three times at 5 min intervals to avoid sensitization and the withdrawal thresholds were averaged and recorded.

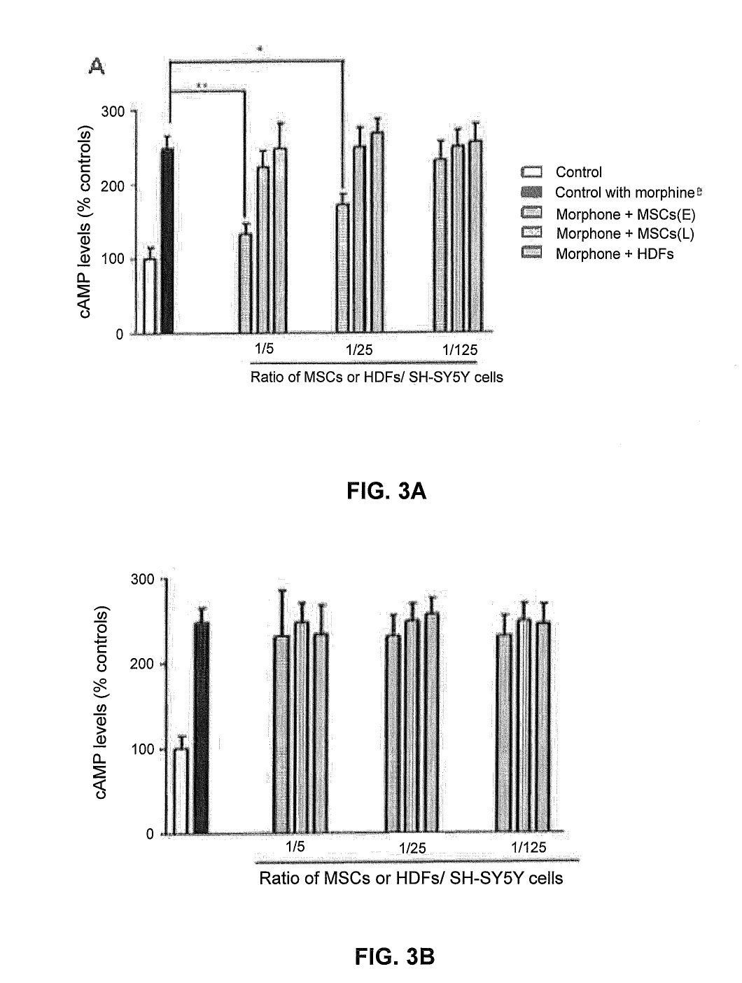

Thermal pain sensitivity. The withdrawal thresholds to thermal stimulation were determined using the Basile Plantar test apparatus (Stoelting Company, Wood Dale, Ill.) (FIG. 3B). The animal was loaded into a Perspex enclosure, resting on a pane of glass unrestrained. After a 15 min acclimation period, a movable infrared generator was positioned below the plantar surface of the hindpaw and activated. The infrared generator and a timer measuring the duration of the stimulation stopped automatically upon withdrawal of the paw. The withdrawal threshold was the latency between the activation of the heat source and the paw withdrawal. The intensity of the heat stimulus was adjusted to evoke a withdrawal response in 10-20 seconds in normal animals. A cutoff of 30 seconds was used to prevent potential tissue damage. The procedure was repeated three times at 5 min intervals to avoid sensitization and the withdrawal thresholds were averaged and recorded.

Statistical analysis. Mechanical and thermal threshold values were expressed as means±sem (standard error). A two-way analysis of variance (ANOVA) with repeated measures was used to determine the treatment effects between the two groups in different time intervals. Post hoc paired comparisons were made using Bonferroni's t-test. For all statistical tests, P<0.05 was considered statistically significant. This part of the study was performed by two postdoctoral fellows with advanced training in biostatistics.

### Results

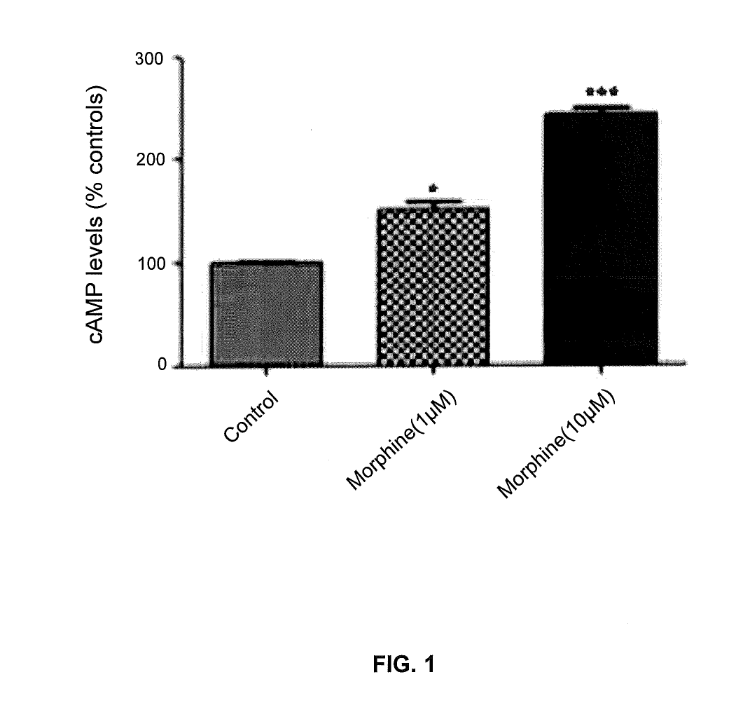

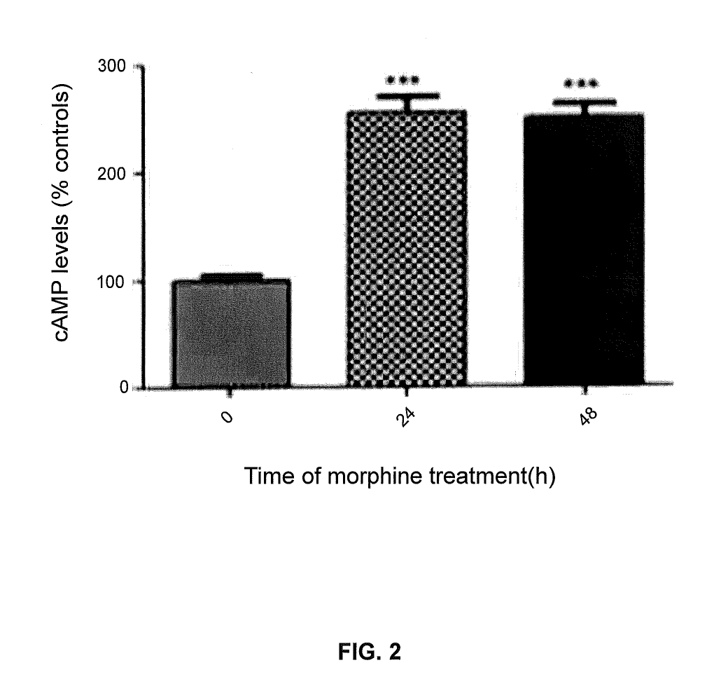

MSCs inhibit the development of morphine-induced tolerance in SH-SY5Y cell model. Exposure of cultured SH-SY5Y cells to 1 μM or 10 μM morphine for 24 h led to significantly increased cAMP levels in the cultured SH-SY5Y cells compared to control cells not treated with morphine (FIG. 1, p<0.05˜p<0.001). The cAMP levels were increased to 150.7±20.6% of control following exposure to 1 μM morphine, and to 241.6±19.6% of control by 10 μM morphine, respectively. The cAMP levels of SH-SY5Y cells treated by 10 μM morphine were then measured at different time points (0, 24 h, and 48 h) (FIG. 2). Treatment of SH-SY5Y cells with morphine caused significant increase of cAMP in SH-SY5Y cells at 24 and 48 h (FIG. 2, p<0.001).

SH-SY5Y cells were pretreated with 10 μM morphine for 24 h, co-cultured for an additional 36 h with MSCs or HDFs at various cell ratios, either with or without cell-to-cell contact, and cAMP levels were measured. Increased cAMP in morphine treated SH-SY5Y cells was significantly attenuated by co-culturing with hMSCs of early passage, i.e five or fewer passages (P) with cell-to cell-contact (p<0.05 at a ratio of 1/25 for hMSCs/SH-SY5Y cells and p<0.01 at a ratio of 1/5 for hMSCs/SH-SY5Y cells). These results show that inhibition of the development of morphine tolerance in SH-SY5Y cells increases with increasing ratios of hMSCs:SH-SY5Y cells (FIG. 3A). HDFs and hMSCs of late passages (P>10) showed no detectable inhibitory effect of morphine-induced increase in cAMP (FIG. 3A). HDFs, which were used as a control, are terminally differentiated and lack differentiation and colony-forming potential. No inhibition of morphine-induced increase in cAMP was detected in the co-cultures without physical contact between SH-SY5Y cells and hMSCs (FIG. 3B). These results suggest that the anti-tolerance effect is specific to hMSCs and may be attributed to the interaction of cell-to-cell contact in the co-cultures between SH-SY5Y cells and hMSCs. The proliferation rate of hMSCs of early passages, one of the characteristics of stem cells, is significantly higher than that of hMSCs of late passages (P>10, data not shown), and hMSCs of late passages may have differentiated or gradually lost their “sternness” during long period of in vitro culture. This suggests that the “sternness” of hMSCs at the early stage may also play a role in the anti-tolerance effect.

Opioid therapy is a cornerstone of pain management. However, long-term use of opioids for the pain management is hampered by analgesic tolerance, which requires escalating doses of drug to maintain pain relief at the same level. Other negative health consequences related with the long-term use of opioids include cognitive impairment, drug abuse, and addiction. Although the molecular mechanisms of opioid tolerance are still unclear, the most observed correlative biochemical adaptation both in vitro and in vivo is the upregulation of the cAMP system, including increased intracellular cAMP levels, adenylate cyclase supersensitization, and other chronic changes that involve activation of transcription factors leading to alterations in protein expression. Thus, cAMP system upregulation after chronic opioids has been proposed as a biochemical mediator underlying chronic effects of opioids and a cellular hallmark to study morphine tolerance. Experiments to generate analgesic and anti-tolerant cells from hMSCs by an approach of cell reprogramming, naive hMSCs at the early passages (≦passage 5) showed significant inhibitory effect on the development of morphine-induced tolerance in differentiated SH-SY5Y cells, i.e., significantly attenuated morphine-induced cAMP up-regulation (FIG. 3A), suggesting hMSCs may have therapeutic potential for the opioid tolerance treatment in clinics.

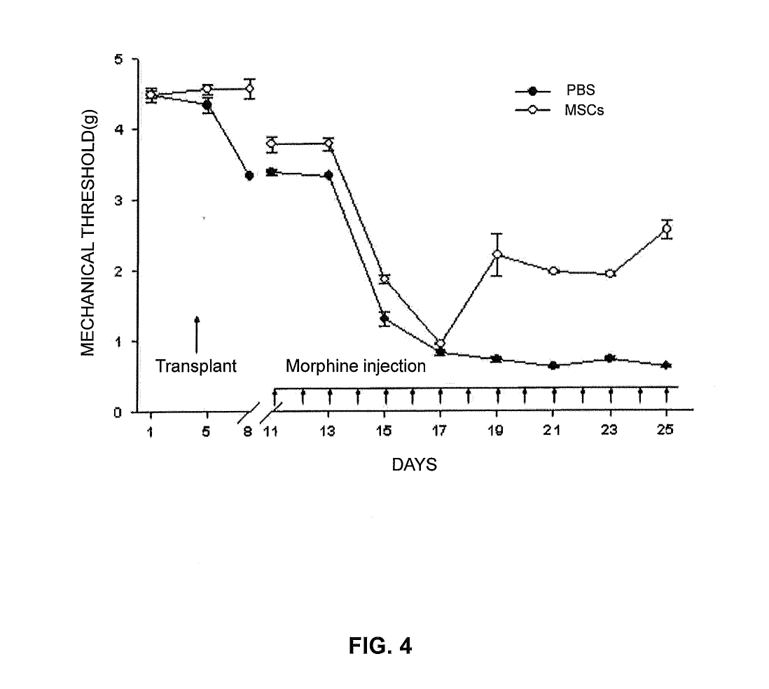

Analgesic effect of MSC transplantation. The mean withdrawal thresholds to mechanical stimulation in the MSC group were significantly higher at Day 8 (3 days after the transplantation at day 5), compared to the PBS control group (t=9.115, p<0.001) (FIG. 4). This effect was still significant at day 11 before the initiation of daily morphine injections (t=2.90, p<0.01). Compared to the baseline values at day 1, the withdrawal thresholds were significantly reduced in the control group at day 8 (t=9.395, p<0.001). In contrast, the thresholds in the MSC group did not differ from the baseline values (t=0.530, p=1).

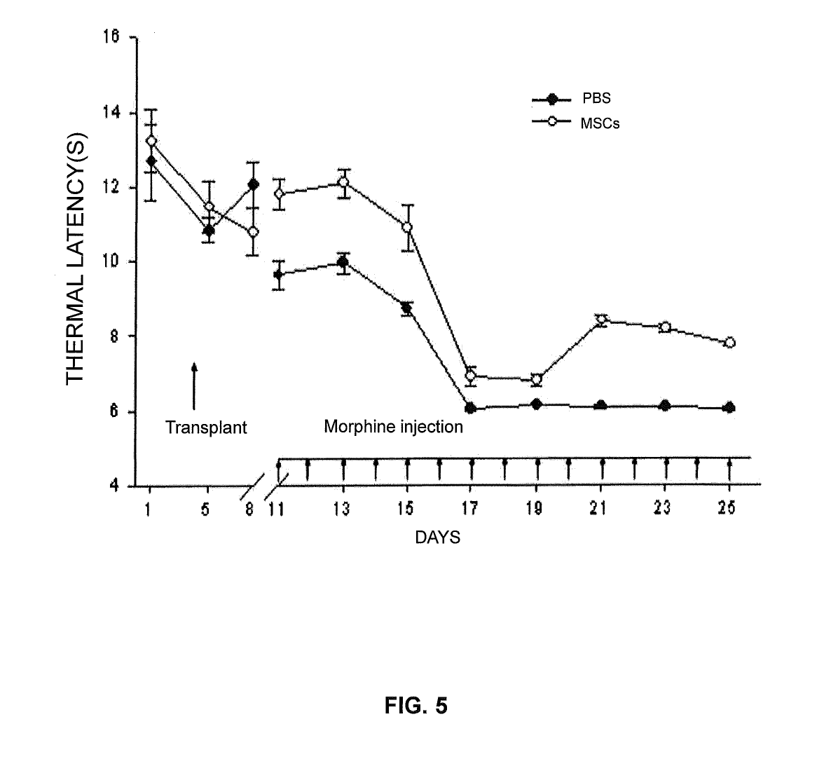

Similarly, the mean withdrawal thresholds to thermal stimulation in the MSC group were significantly higher at days 11 and 13, compared to the control group (day 11, t=6.080, p<0.001; day 13, t=6.010, p<0.001) (FIG. 5). Compared to the baseline values at day 1, the withdrawal thresholds were significantly reduced at days 11 and 13 in the control group (day 11, t=8.947, p<0.001; day 13, t=8.034, p<0.001). In contrast, the thresholds in days 11 and 13 of the MSC group did not differ from the baseline values (p>0.05). These data revealed an analgesic effect of MSC transplantation, which did not compromise the protective role of pain.

Taken together, these data revealed an analgesic effect of MSC transplantation. This effect prevented the reduction of the withdrawal thresholds observed in the control group. Importantly, the withdrawal thresholds were never elevated above the baseline control values after the transplantation, indicating that the analgesic effect did not compromise the animals' ability to respond to noxious stimulation to protect them.

Morphine-induced tolerance. Daily morphine injections gradually and significantly reduced the withdrawal thresholds to mechanical stimulation within five days in the control group, indicating the development of morphine tolerance (FIG. 4).

Compared to the pre-morphine injection value at day 11, the mean mechanical withdrawal thresholds were dramatically decreased only 4 days after the start of daily morphine injections (Day 15, t=15.639, p<0.001) (FIG. 4). Further decreases of mean mechanical withdrawal thresholds were consistently observed throughout the rest of the experiments up to Day 25 with continued daily morphine injections, leading to extremely low withdrawal thresholds (high pain sensitivity).

Similarly, compared to the pre-morphine injection values at day 11, the withdrawal thresholds to thermal stimulation in the control group were also significantly reduced at Day 17 and thereafter (p<0.05) (FIG. 5). Clearly, morphine tolerance had developed with repeated daily morphine injections, resulting in extremely low withdrawal thresholds to nociceptive mechanical or thermal stimulation in the control group.

Anti-tolerance effect of MSC transplantation. Daily morphine injections also gradually and significantly reduced the withdrawal thresholds in the MSC group, again indicating morphine tolerance (FIGS. 4 and 5). However, the reduction of the withdrawal thresholds in the MSC group was significantly less compared to the control group. The mean mechanical and thermal withdrawal thresholds were consistently higher throughout the whole course of daily morphine injection. Furthermore, the reduction of the mechanical withdrawal thresholds was substantially reversed in Day 19 and thereafter. The mean withdrawal thresholds tom mechanical stimulation were significantly higher in Days 19, 21, 23, and 25, compared with day 17 (t=8.001-13.093, 10.183, p<0.001) (FIG. 4). Similarly, the mean withdrawal thresholds to thermal stimulation in days 21, 23, and 25 were also higher than those in days 17 and 19, although to less extents (FIG. 5). These data clearly indicate that morphine-induced tolerance was substantially attenuated by the MSC transplantation.

The results show that intrathecal transplantation of MSCs enhances opioid analgesic effect and reduces morphine tolerance in rats. For the first time, a significant analgesic and anti-tolerance effect of MSCs was demonstrated. Both the mechanical and thermal withdrawal thresholds were increased in the MSC group compared to the control group after the transplantation (FIGS. 4 and 5). This analgesic effect of MSC transplantation was so powerful that it effectively prevented the significant reductions of the mechanical and thermal withdrawal thresholds that were observed in the control animals. It is important to note that the withdrawal thresholds were never elevated about the baseline values, suggesting that the protective, physiological role of pain sensation was not compromised by MSC transplantation. The animals maintained their normal ability to respond to noxious stimulation to protect their body from injuries.

Morphine tolerance was consistently and reliably induced by daily morphine injections within 5 to 7 days in the control group. This tolerance was reflected by the progressive reduction of mechanical and thermal withdrawal thresholds throughout the whole two-week course of daily morphine injections. This reduction was observed consistently while the dose of the daily morphine injections was kept constant. This result made it possible for us to test anti-tolerance strategies in an animal model before conducting clinical trials in humans.

A potent anti-tolerance effect of MSC transplantation was observed. Compared to the control group, both the mechanical and thermal withdrawal thresholds were substantially and consistently increased in the MSC group (FIGS. 4 and 5). This is particularly evident for the mechanical withdrawal thresholds. The effect on thermal withdrawal threshold was similar but less prominent. The difference in amplitude of the effect between the mechanical and thermal responses is likely due to the fact that the mechanical nociception and thermal nociception are mediated by different nociceptors and neural pathways. Additionally, the analgesic effects of morphine through mechanical and thermal nociceptors are mediated by a different set of subtypes of opioid receptors, leading to different degrees of anti-tolerance effects of MSC transplantation.

It is understood that the foregoing detailed description and accompanying examples are merely illustrative and are not to be taken as limitations upon the scope of the invention, which is defined solely by the appended claims and their equivalents.

What is claimed is:

1. A method of treating pain in a subject comprising administering an effective pain-reducing number of mesenchymal stem cells to the subject.

2. The method of claim 1 wherein the mesenchymal stem cells are harvested from bone marrow.

3. The method of claim 2 wherein the bone marrow is the autologous bone marrow.

4. The method of claim 2, wherein the mesenchymal stem cells are expanded in cell culture prior to administration.

5. The method of claim 1, wherein the mesenchymal stem cells are administered into an intrathecal space of the subject.

6. The method of claim 1 wherein the mesenchymal stem cells are administered two or more times.

7. The method of claim 1 further comprising treating the subject with an effective amount of one or more opioids.

8. The method of claim 7 wherein the opioid is morphine.

9. The method of claim 7 wherein the mesenchymal stem cells are administered to the subject prior to treating with the opioids.

10. The method of claim 7 wherein the effective amount of the one or more opioids is lower than the amount required to achieve the same effect in the absence of the transplanted mesenchymal stem cells.

11. A method of attenuating opioid tolerance in a subject comprising administering a number of mesenchymal stem cells effective to prevent or attenuate opioid tolerance.

12. The method of claim 11 wherein the mesenchymal stem cells are administered to the subject prior to treating with the opioids.

13. The method of claim 11 wherein the mesenchymal stem cells are administered two or more times.

14. The method of claim 11 wherein the mesenchymal stem cells are injected into an intrathecal space of the subject.

15. The method of claim 11 wherein the mesenchymal stem cells are harvested from bone marrow.

16. The method of claim 15 wherein the bone marrow is the subject's bone marrow.

17. The method of claim 16 1 wherein the mesenchymal stem cells are expanded in cell culture after harvesting from bone marrow.

18. A method of treating pain in a subject comprising administering to the subject a number of mesenchymal stem cells effective to reduce pain and one or more opioids.

19. The method of claim 18 wherein the mesenchymal stem cells are administered to the subject prior to treating with the opioids.

20. The method of claim 18 wherein the mesenchymal stem cells are administered two or more times.

21. The method of claim 18 wherein the mesenchymal stem cells are injected into an intrathecal space of the subject.

22. The method of claim 18 wherein the mesenchymal stem cells are harvested from bone marrow.

23. The method of claim 22 wherein the bone marrow is autologous bone marrow.

24. The method of claim 23 wherein the mesenchymal stem cells are expanded in cell culture prior to administration.
